(19) United States
(12) Patent Application Publication
(10) Pub. No.: US 2013/0196418 A1
(43) Pub. Date: Aug. 1, 2013

(54) ECOSYSTEM RECOVERY FROM IONIZING RADIATION

(71) Applicant: Vishal Shah, Oakdale, NY (US)

(72) Inventor: Vishal Shah, Oakdale, NY (US)

(21) Appl. No.: 13/827,976

(22) Filed: Mar. 14, 2013

Related U.S. Application Data
(62) Division of application No. 13/474,799, filed on May 18, 2012.
(60) Provisional application No. 61/487,999, filed on May 19, 2011.

(51) Int. Cl. C12N 1/20

(52) U.S. Cl. CPC C12N 1/20
USPC 435/252.1

(57) ABSTRACT

A method for aiding an ecosystem in recovering from the effects of exposure to radiation, comprising introducing microorganisms into the ecosystem, the microorganisms preferably replacing native microorganisms destroyed, killed, or reduced in number by the exposure to radiation. A method for aiding an ecosystem in recovering from the effects of exposure to radiation by, prior to the ecosystem being exposed to radiation, cataloging the microorganisms in the soil of the ecosystem; after the ecosystem being exposed to radiation, cataloging the microorganisms in the soil of the ecosystem; and introducing into the ecosystem microorganisms before and/or after the ecosystem has been exposed to radiation. The microorganisms introduced into the ecosystem are microorganisms present in the ecosystem prior to exposure to radiation or microorganisms equivalent to the microorganisms present in the ecosystem prior to exposure to radiation.

### CROSS REFERENCE TO RELATED APPLICATIONS

This patent application is a divisional of and claims the benefit of U.S. patent application Ser. No. 13/474,799 having a filing or 371(c) date of 18 May 2012, which is the non-provisional of and claims the benefit of U.S. Provisional Patent Application No. 61/487,999 having a filing date of 19 May 2011.

### STATEMENT OF GOVERNMENT INTEREST

This invention was made with US government support under contract no. CBET-1028438 awarded by the US National Science Foundation. The US government has certain rights in the invention.

### BACKGROUND OF THE INVENTION

1. Technical Field

The present invention generally relates to the technical field of the recovery of ecosystems after exposure to ionizing radiation, and more specifically relates to the field of assisting the recovery of ecosystems after exposure to ionizing radiation by introducing radiation sensitive microorganisms into the ecosystem prior to or after the exposures of the ecosystem to radiation.

2. Prior Art

High concentrations of radioactive isotopes can find their way into the environment mainly during the storage of high-level radioactive wastes (1). High-level radioactive wastes include the spent nuclear reactor fuels generated by nuclear power plants, the wastes generated during reprocessing of these fuels, and the wastes generated during the development of nuclear weapons (2). High-level radioactive wastes are stored in spent fuel pools or in dry cask storage facilities, pending its eventual disposition in a national repository site. During this disposal period, the contamination of the natural ecosystem by accidental slow release of high-level radioactive wastes always remains a possibility (3,4). Occasional release of radioactive isotopes into the environment also happens during accidents involving nuclear fuel rods. Examples of such accidents include the Chernobyl disaster in 1986 and the recent damage to the nuclear power plants in Okuma, Japan in 2011.

The exposure of an ecosystem to radiation can cause flora and fauna, microorganisms and macroorganisms, to be killed or destroyed, to be reduced in number or decimated, or to leave the exposed ecosystem. This phenomenon has been noted in radiation-exposed ecosystems. The reduction in one type of organism can affect the health and population of other types of organisms. It is well known that various organisms depend on other organisms to thrive. Exposure of an ecosystem to radiation can negatively impact the population of various different types of organisms.

Thus, it can be seen that there is need for improved and new methods for aiding in the recovery of an ecosystem after exposure to radiation. It is to this need that the present invention is directed

### REFERENCES

1. R. G. Riley, J. M. Zachara, F. J. Wobber. Chemical contaminants on DOE lands and selection of contaminant mixtures for subsurface science research. US Department of Energy, Office of Energy Research, Subsurface Science Program (1992).
2. Disposition of high-level radioactive waste through geological isolation. Development, current status, and technical and policy challenges. National academy press, Washington D.C. (1999). Available at 222.nap.edu/catalog/9674.html
3. M. J. Daly. Current Opinion Biotechnol. 11, 280 (2000).
4. J. K. Fredrickson et al., Appl. Environ. Microbiol. 70, 4230 (2004).
5. M. Al-Bachir, M. A. Al-Adawi, M. Shamma, Bioresource Technol. 90, 139 (2003).
6. N. P. McNamara, H. I. J. Black, N. A. Beresford, N. R. Parekh, Appl Soil Ecol. 24, 117 (2003).
7. N. P. McNamara, R. I. Griffiths, N. A. Beresford, M. J. Bailey, A. S. Whiteley, Appl. Soil Ecol. 37, 1 (2007).
8. R. J. Melcher, S. E. Apitz, B. B. Hemmingsen, Appl. Environ. Microbiol. 68, 2858 (2002).
9. S. Fuma, N. Ishii, H. Takeda, K. Doi, I. Kawaguchi, S. Shikano, N. Tanaka, Y. Inamori, J. Environ. Radioact. 101, 915 (2010).
10. G. M. Woodwell, Rad. Bot. 3, 125 (1963).
11. R. Stalter, D. Kincaid, American J. Bot. 96, 2206 (2009).
12. G. M. Woodwell, L. N. Nukker, Science. 139, 222 (1963).
13. A. Nocker, A. K. Camper, Appl. Environ. Microbiol. 72, 1997 (2006).
14. E. Dadachova, A. Casadevall. Current Opinion Microbiol. 11, 525 (2008).
15. N. V. Mironenko, I. A. Alekhina, N. N. Zhdanova, S. A. Bulat, Ecotoxicol. Environ. Safety. 45, 177 (2000).
16. L. St Cyr Jerilynn, M. Kambhampati, T. Green, J. Dvorak Application of Near-Edge X-Ray Absorption Fine Structure Spectroscopy to Detect Nitrogen in Solar Farm Soils in Long Island, N.Y. (2010). Available at http://www.bnl.gov/esd/wildlife/PDF/Research_papers/Stcyr_paper_{—}2010.pdf
17. M. Monib, M. N. Zayed, J. Appl. Microbiol. 26, 35 (2008).
18. W. de. Boer, L. B. Folman, R. C. Summerbell, L. Boddy FEMS Microbiol. Rev. 29, 795 (2005).
19. S. E. Dowd et al., BMC Microbiol. 8, 43 (2008).
20. T. R. Callaway et al., J. Animal Sci. 88, 3977 (2010).
21. S. M. Finegold et al., Anaerobe 16, 444 (2010).
22. V. Gontcharova, E. Youn, R. D. Wolcott, E. B. Hollister, T. J. Gentry, Open Microbiol J. 4, 47 (2010).
23. S. E. Dowd, J. Zaragoza, J. R. Rodriguez, M. J. Oliver, P. R. Payton, BMC Bioinformatics 6, 93 (2005).
24. D. M. Smith et al., BMC Med Genomics 3, 1 (2010).
25. S. S. Handl et al., FEMS Microbiol. Ecol. In Press (2011).
26. C. M. Lozupone et al., ISME J. 5, 169 (2010).
27. P. D. Schloss et al., Appl Environ Microbiol. 75, 7537 (2009).

### BRIEF SUMMARY OF THE INVENTION

Briefly, the present invention is a method of aiding the recovery of an ecosystem from exposure to radiation, particularly ionizing radiation, by introducing microorganisms into the ecosystem prior to the ecosystem being exposed to radiations and/or by reintroducing microorganisms destroyed by the radiation back into the ecosystem after the ecosystem has been exposed to radiation. Exposure of an ecosystem to ionizing radiation remains a possibility either due to accidents involving nuclear facilities and/or nuclear fuel rods or with high-level radioactive wastes, resulting in contamination of an ecosystem with radiation. The 2011 tsunami affecting Japan caused such a radiation contamination by damaging a nuclear reactor site. While the short and long term effect of ionizing radiation on higher eukaryotes has been well documented, we do not have an understanding on the recovery of microbial community post radiation.

In one embodiment of the present invention, ecosystems, such as the site within Long Island Pine Barrens Forest that was radiated from 1961 to 1978 with gamma rays, which have not yet recovered from the effects of radiation, can be aided in recovery by reintroducing microorganisms destroyed by the radiation back into the ecosystem. In another embodiment of the present invention, the effect of radiation on an ecosystem potentially can be reduced by introducing microorganisms into the ecosystem prior to exposure of the ecosystem to radiation. This method can further comprise cataloging the microorganisms, flora and/or fauna in the ecosystem prior to and after the ecosystem has been exposed to radiation such that native microorganisms or the equivalent can be introduced or reintroduced into the ecosystem.

The current vegetation type in such ecosystems contaminated by radiation often varies as one moves away from the source of the ionizing radiation, with the region closest to the source typically having no vegetation. TEFAP analysis of the soil pre- and post-ionizing radiation suggests that the difference in vegetation originates from the difference in microbial community present in the soil. The absence of the ionizing radiation sensitive microorganisms that are essential for the growth of the vegetation, and the presence of fungi that delays the re-colonization of the soil by the essential radiation sensitive microorganisms makes the recovery of the ecosystem more difficult. To allow faster recovery of the ecosystem from future ionizing radiation exposures in high risk areas, the present invention contemplates studying and archiving the radiation sensitive microorganisms in an ecosystem that are essential for proper health of the ecosystem. Should the ionizing radiation find its way into such high-risk areas, artificial reintroduction of the essential microorganisms into the ecosystem could allow a faster recovery of the ecosystem.

These features, and other objects, features and advantages of the present invention will become more apparent to those of ordinary skill in the art when the following detailed description of the preferred embodiments is read in conjunction with the appended figures.

### BRIEF DESCRIPTION OF THE DRAWINGS

FIG. 1a shows a vegetation survey of the Gamma Forest in 1978.

FIG. 1b shows a vegetation survey of the Gamma Forest in the summer of 2010 revealing that the forest can be distinctly divided into five different circular zones. The sampling location for the microbial study is shown in FIG. 1b.

FIG. 2 provides visualization and hierarchal clustering of the samples in the Gamma Forest based upon the top 50 genera using Dual Hierarchal clustering of individual samples. Top 50 genera based upon the average relative percentages in each sample were evaluated using Ward's minimum variance and Manhattan distance. Pre-radiation samples are denoted with C and post-radiation samples denoted with R. There is clear separation of the pre- and post-radiation samples based upon the top 50 genera.

FIG. 3 shows that based upon Unifrac analysis and principal component analysis pre-radiation (blue) and radiation (red) samples are clearly and significantly (p<0.001) grouped in three dimensional space. This Unifrac principle component analysis is a three dimensional PCA and illustrates a clear differentiation of the two groups of samples.

### DETAILED DESCRIPTION OF PREFERRED EMBODIMENTS

The ionizing radiation from radioactive isotopes is known to cause irreparable damage to cells. Of relevance to the current invention are the effects of ionizing radiation on the microorganisms in the environment, such as, for example, microorganisms in a specific ecosystem. Microorganisms play a crucial role in maintaining the health and function of an ecosystem, and any change in microbial diversity can influence the quality and health of an ecosystem. Several studies have shown that ionizing radiation immediately changes the microbial population in soil, sediment and water systems (5-9). However, literature on the long term effect of ionizing radiation on microbial communities does not exist. The question of whether microbial populations exposed to ionizing radiation recover in natural environments remains unanswered.

The Gamma Forest was a radiation facility established in 1961 within the Long Island Pine Barrens Forest, N.Y., US, to provide opportunity for systematic study of the effects of ionizing radiation on a terrestrial ecosystem and its components (10). The source of radiation used during the experiment was a ¹³⁷cesium (9500 curies) gamma emitter (10). In the study, the radiation source was raised remotely via a mast located in the center of a field within the forest and the field was exposed to the radiation for 20 hours per day for 18 years until 1978 (11). Rates of radiation exposure around the source varied from several thousand roentgens per day within a few meters of the source to about 2 roentgens per day at 130 meters from the source (10). It now has been more than 30 years since the exposure of the forest to ionizing radiation was terminated. During this post-radiation exposure period, the forest has been placed under limited access, thereby preventing any influence of human activity on the recovery. The current study provides evidence that even after 30 years since the exposure, the ecosystem within the Gamma Forest has not recovered from the effect of ionizing radiation.

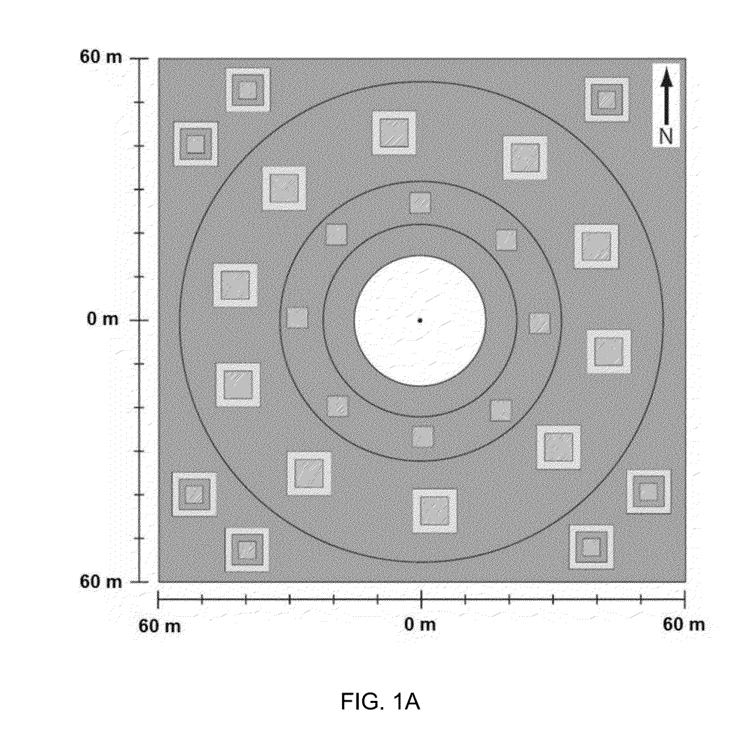

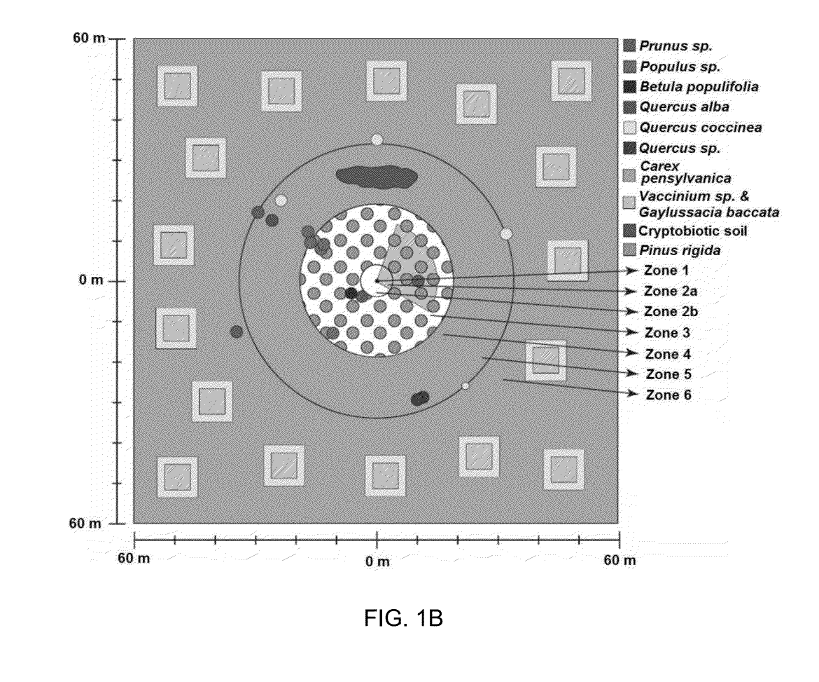

A vegetation survey of the Gamma Forest in summer of 2010 revealed that the forest can be distinctly divided into different circular zones (FIG. 1b), with Zone 1 being the innermost zone closest to the radiation source. Even after 30 years, there is still no vegetation growing in the area, Zone 1, where the ¹³⁷cesium source was located. Zone 2 could be divided into two areas. An area in the northeast quadrant, Zone 2a, has a growth of blueberries (Vaccinium sp.) and black huckleberries (Gaylussacia baccata (Wangenh) Koch). The remaining area in Zone 2 is an empty patch of soil with no vegetation (Zone 2b). Zone 3 is primarily comprised of 68 pitch pine trees (Pinus rigida P. Mill.) scattered uniformly between 5 and 20 m radius. Pine needles cover the floor of the forest uniformly out to 18 m from the source of radiation, after which there is an immediate transition to a total absence of the needles. Moss and lichens appear after a distance of 20 m from the source of radiation. Magnolia virginiana L. (sweet bay) is scattered at the interface of Zones 3 and 4. Pennsylvania sedge (Carex pensylvanica Lam.) and scattered young pitch pine trees (9 years old in 2010, based on ring formation) are found in Zone 4. Blueberry shrubs are also randomly scatted within Zone 4. Zone 6 has the composition of a normal Pine Barrens Forest (Pennsylvania sedge, blueberry, huckleberry and scarlet oak trees (Quercus coccinea Münchh). Between Zones 4 and 6 is a small zone (Zone 5) consisting primarily of Pennsylvania sedge, blueberry and huckleberry.

When one observes the vegetation that survived the ionizing radiation, five distinct zones were observed (FIG. 1b). No vegetation was observed in the immediate 15 m radius around the source of ionizing radiation (Zone 1). Zone 2 showed the presence of Pennsylvania sedge, and Zone 3 had the presence of blueberry and huckleberry, in addition to Pennsylvania sedge. The trees were seen to survive from approximately 35 m outward with oak trees being the major trees in (Zone 4) and pine trees seen in Zone 5 along with oak trees.

Comparison of FIG. 1a and FIG. 1b yields a conclusion that the regions that received a high dosage of ionizing radiation have yet to recover from the effects of radiation. While it is not clear why even after 30 years in an undisturbed area within a forest the vegetation has not recovered, surprisingly there is no correlation between the radiation dosage and the type of vegetation. Zone 3, which received a higher dosage of radiation than Zone 4, has older pitch pine trees. This is opposite to what would be expected, considering the high sensitivity of the pitch pine trees to gamma radiation (12).

The difference in vegetation originates from the difference in microbial community present in the soil. Growth of vegetation requires association with numerous microbial populations. Radiation of the forest between 1961 and 1978 would have killed all the microorganisms except the most ionizing radiation resistant microorganisms. The region nearest the radiation source had only the most radiation-resistant microorganisms in the soil remaining viable, while at greater distances, the decreasing radiation intensity allowed the more sensitive microorganisms to remain viable, along with the resistant ones. Among the more sensitive microorganisms could be the key organisms required for the growth of higher plants. For example, the presence of the more sensitive microorganisms could be important to the presence, growth, and/or health of higher order plants, and reintroducing the more sensitive microorganisms to this ecosystem could result in the reemergence of higher order plants.

In the present invention, we obtained the surface soil (0-25 cm) from different zones of the forest and radiated the samples to 1.8 kGy of gamma radiation. Pre-radiated and post-radiated soil samples were analyzed using tag-encoded FLX amplicon pyrosequencing (TEFAP, www.researchandtesting.com) to identify the bacteria and fungi present, respectively. Ethidium Monoazide treatment was utilized to allow the isolation of DNA only from the viable cells (13). The results from the pyrosequencing are shown in Table 1 and Table 2.

As can be seen, radiation-sensitive bacteria occupied maximum percentage of the microbial community in the pre-radiated samples (Table 1). Among the microorganisms that are sensitive to ionizing radiation are the organisms involved in the nitrogen cycle: Rhizobiaceae, Bradyrhizobiaceae and Nitrosomonadaceae (Table 1). The percentage of radiation-resistant bacteria was so low in the pre-radiation samples that they were below detection of the TEFAP assay at the relatively high level of resolution utilized in the current study.

TABLE 1
Percentage of 16s rDNA sequences of the bacterial families present in the soil before (C) and after
radiation (R) to 1.8 kGy gamma radiation. The soil sampling locations are shown in FIG. 1b.

| | Zone | | Zone | | Zone | | Zone | | Zone | | Zone | | Zone | |
|---|---|---|---|---|---|---|---|---|---|---|---|---|---|---|
| | 1 | | 2a | | 2b | | 3 | | 4 | | 5 | | 6 | |
| Name | C | R | C | R | C | R | C | R | C | R | C | R | C | R |
| Acetobacteraceae | 8 | 1 | 7 | 1 | 7 | 2 | 6 | 4 | 5 | 0 | 2 | 1 | 3 | 1 |
| Acidobacteriaceae | 24 | 11 | 20 | 7 | 17 | 5 | 20 | 8 | 23 | 2 | 23 | 6 | 33 | 5 |
| Alicyclobacillaceae | 0 | 0 | 0 | 0 | 0 | 0 | 0 | 0 | 0 | 0 | 0 | 10 | 0 | 5 |
| Bacillales (family) | 0 | 0 | 0 | 0 | 0 | 0 | 0 | 0 | 0 | 1 | 0 | 3 | 0 | 0 |
| Beijerinckiaceae | 1 | 0 | 0 | 0 | 1 | 0 | 0 | 1 | 1 | 0 | 2 | 1 | 1 | 4 |
| Bradyrhizobiaceae | 6 | 2 | 5 | 1 | 7 | 1 | 4 | 3 | 10 | 1 | 4 | 2 | 2 | 1 |
| Burkholderiaceae | 5 | 2 | 12 | 0 | 8 | 0 | 4 | 0 | 5 | 0 | 26 | 0 | 5 | 0 |
| Caldilineaceae | 1 | 25 | 0 | 19 | 2 | 23 | 0 | 11 | 0 | 25 | 0 | 23 | 0 | 23 |
| Caulobacteraceae | 2 | 1 | 3 | 0 | 1 | 0 | 3 | 0 | 1 | 0 | 0 | 1 | 2 | 0 |
| Desulfobacteraceae | 0 | 0 | 0 | 0 | 0 | 0 | 0 | 0 | 0 | 0 | 2 | 0 | 0 | 0 |
| Gemmatimonadaceae | 0 | 0 | 0 | 1 | 1 | 0 | 0 | 0 | 0 | 4 | 0 | 0 | 0 | 1 |
| Holophagaceae | 13 | 8 | 12 | 4 | 14 | 6 | 15 | 11 | 10 | 4 | 12 | 6 | 10 | 2 |
| Hydrogenophilaceae | 0 | 0 | 0 | 0 | 0 | 16 | 0 | 0 | 0 | 0 | 0 | 0 | 0 | 0 |
| Hyphomicrobiaceae | 3 | 1 | 1 | 0 | 2 | 0 | 2 | 1 | 1 | 1 | 1 | 1 | 2 | 1 |
| Ktedonobacteraceae | 0 | 3 | 0 | 5 | 0 | 12 | 0 | 6 | 0 | 8 | 0 | 1 | 0 | 0 |
| Ktedonobacteria (family) | 0 | 4 | 0 | 3 | 0 | 3 | 0 | 4 | 0 | 2 | 0 | 2 | 0 | 0 |
| Methylobacteriaceae | 0 | 0 | 0 | 19 | 0 | 0 | 0 | 10 | 0 | 18 | 0 | 1 | 0 | 26 |
| Nitrosomonadaceae | 8 | 1 | 9 | 2 | 9 | 3 | 16 | 12 | 11 | 3 | 10 | 14 | 12 | 15 |
| Rhizobiaceae | 5 | 1 | 5 | 1 | 6 | 1 | 8 | 2 | 8 | 1 | 4 | 4 | 6 | 2 |
| Rhodospirillaceae | 1 | 0 | 3 | 0 | 2 | 0 | 1 | 0 | 2 | 0 | 0 | 0 | 2 | 0 |
| Solibacteraceae | 3 | 0 | 4 | 0 | 1 | 0 | 2 | 0 | 2 | 0 | 2 | 0 | 3 | 0 |
| Thermosporotrichaceae | 1 | 30 | 0 | 27 | 2 | 17 | 0 | 13 | 0 | 22 | 0 | 14 | 0 | 2 |
| Xanthomonadaceae | 2 | 1 | 3 | 0 | 4 | 1 | 3 | 1 | 5 | 0 | 5 | 1 | 3 | 1 |

TABLE 2
Percentage of 18s rDNA sequences of fungi families present in the soil before (C) and after
radiation (R) to 1.8 kGy gamma radiation. The soil sampling locations are shown in FIG. 1.

| | Zone | | Zone | | Zone | | Zone | | Zone | | Zone | | Zone | |
|---|---|---|---|---|---|---|---|---|---|---|---|---|---|---|
| | 1 | | 2a | | 2b | | 3 | | 4 | | 5 | | 6 | |
| Name | C | R | C | R | C | R | C | R | C | R | C | R | C | R |
| Agaricales (family) | 0 | 0 | 0 | 0 | 0 | 2 | 0 | 0 | 0 | 0 | 0 | 0 | 0 | 0 |
| Ascomycota (family) | 2 | 0 | 1 | 3 | 1 | 0 | 1 | 0 | 17 | 0 | 1 | 0 | 4 | 0 |
| Atheliaceae | 2 | 0 | 1 | 7 | 0 | 0 | 0 | 0 | 0 | 0 | 0 | 0 | 0 | 0 |
| Cantharellales (family) | 0 | 0 | 0 | 0 | 13 | 0 | 0 | 0 | 0 | 0 | 0 | 0 | 0 | 0 |
| Cephalothecaceae | 0 | 0 | 0 | 0 | 0 | 0 | 0 | 0 | 0 | 0 | 3 | 10 | 0 | 0 |
| Chaetosphaeriaceae | 0 | 0 | 1 | 0 | 0 | 0 | 0 | 0 | 2 | 0 | 0 | 0 | 1 | 0 |
| Chaetothyriales (family) | 3 | 0 | 4 | 0 | 0 | 12 | 0 | 0 | 0 | 0 | 0 | 0 | 0 | 0 |
| Clavicipitaceae | 0 | 0 | 0 | 0 | 0 | 0 | 1 | 0 | 14 | 1 | 0 | 0 | 2 | 0 |
| Cordycipitaceae | 0 | 0 | 0 | 0 | 0 | 18 | 0 | 0 | 0 | 0 | 0 | 0 | 0 | 0 |
| Corticiaceae | 0 | 0 | 4 | 5 | 0 | 0 | 2 | 5 | 0 | 0 | 0 | 0 | 1 | 0 |
| Cortinariaceae | 0 | 0 | 0 | 0 | 0 | 0 | 0 | 0 | 0 | 0 | 4 | 0 | 0 | 0 |
| Dermateaceae | 0 | 12 | 5 | 8 | 1 | 0 | 3 | 0 | 0 | 0 | 4 | 6 | 2 | 0 |
| Dothideomycetes (family) | 0 | 0 | 0 | 20 | 0 | 0 | 1 | 10 | 0 | 0 | 0 | 0 | 0 | 0 |
| Eremomycetaceae | 0 | 0 | 0 | 0 | 0 | 0 | 0 | 0 | 2 | 0 | 0 | 0 | 0 | 0 |
| Filobasidiales (family) | 0 | 0 | 0 | 0 | 0 | 0 | 5 | 4 | 0 | 0 | 0 | 0 | 0 | 0 |
| Ganodermataceae | 0 | 3 | 0 | 0 | 0 | 0 | 0 | 0 | 0 | 0 | 0 | 0 | 0 | 0 |
| Helotiaceae | 0 | 0 | 1 | 0 | 0 | 0 | 0 | 0 | 0 | 0 | 0 | 0 | 12 | 0 |
| Helotiales (family) | 27 | 0 | 68 | 18 | 31 | 0 | 17 | 6 | 24 | 6 | 33 | 30 | 5 | 8 |
| Herpotrichiellaceae | 13 | 0 | 1 | 0 | 0 | 0 | 3 | 0 | 0 | 0 | 0 | 0 | 1 | 0 |
| Hypocreaceae | 35 | 0 | 0 | 0 | 1 | 0 | 17 | 0 | 2 | 0 | 0 | 0 | 2 | 0 |
| Hypocreales (family) | 0 | 0 | 1 | 0 | 48 | 0 | 0 | 1 | 2 | 88 | 12 | 0 | 1 | 0 |
| Inocybaceae | 0 | 0 | 0 | 0 | 0 | 0 | 2 | 2 | 0 | 0 | 0 | 0 | 0 | 0 |
| Leotiomycetes (family) | 6 | 10 | 1 | 0 | 0 | 0 | 7 | 14 | 0 | 0 | 2 | 0 | 5 | 0 |
| Lycoperdaceae | 0 | 0 | 0 | 0 | 0 | 0 | 5 | 1 | 18 | 0 | 0 | 0 | 2 | 4 |
| Magnaporthaceae | 0 | 0 | 0 | 0 | 0 | 0 | 0 | 0 | 0 | 0 | 0 | 0 | 0 | 1 |
| Malasseziaceae | 0 | 6 | 0 | 0 | 0 | 0 | 0 | 0 | 0 | 0 | 0 | 0 | 0 | 1 |
| Myxotrichaceae | 1 | 0 | 1 | 0 | 0 | 0 | 6 | 13 | 1 | 0 | 3 | 0 | 0 | 0 |
| Pluteaceae | 0 | 0 | 0 | 0 | 0 | 12 | 0 | 0 | 0 | 0 | 0 | 0 | 0 | 0 |
| Pyronemataceae | 0 | 0 | 0 | 0 | 0 | 0 | 8 | 22 | 0 | 0 | 0 | 0 | 0 | 0 |
| Sarcosomataceae | 0 | 0 | 0 | 0 | 0 | 0 | 2 | 3 | 1 | 0 | 0 | 0 | 0 | 0 |
| Sporidiobolales (family) | 0 | 0 | 0 | 0 | 0 | 0 | 9 | 1 | 0 | 0 | 0 | 0 | 0 | 0 |
| Thamnidiaceae | 1 | 0 | 4 | 29 | 1 | 35 | 2 | 12 | 8 | 4 | 34 | 55 | 11 | 86 |
| Tremellales (family) | 4 | 0 | 1 | 0 | 0 | 0 | 3 | 1 | 0 | 0 | 0 | 0 | 21 | 0 |
| Trichocomaceae | 5 | 69 | 5 | 5 | 2 | 18 | 5 | 3 | 7 | 1 | 1 | 0 | 27 | 0 |
| Tricholomataceae | 0 | 0 | 0 | 3 | 0 | 4 | 0 | 0 | 0 | 0 | 0 | 0 | 0 | 0 |

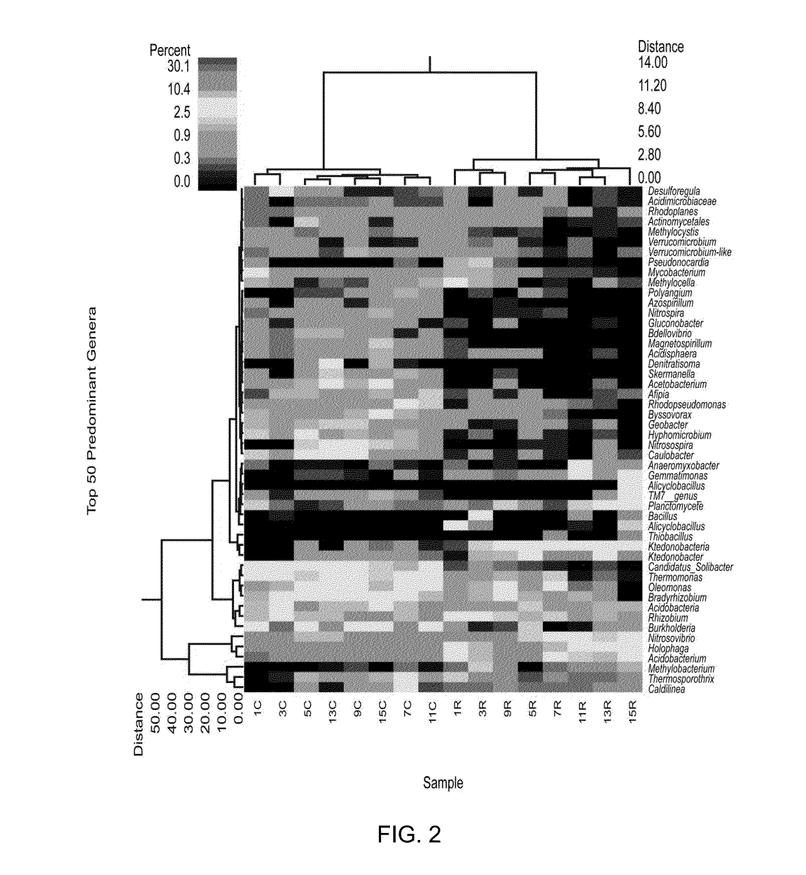

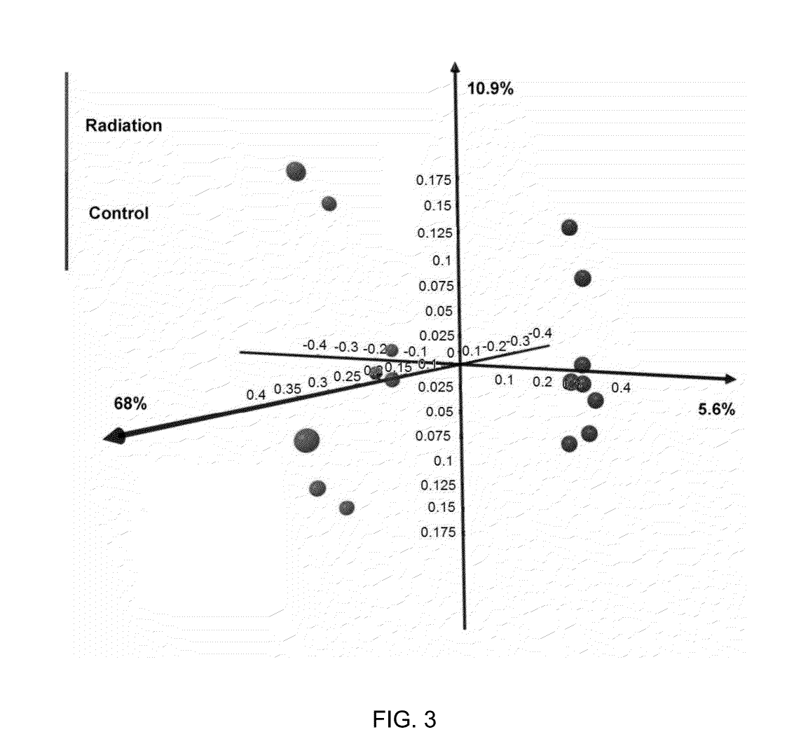

After radiation exposure sensitive organisms declined and radiation-resistant organisms were detected, including the members of Caldineaceae, Methylobacteriaceae and Thermosporotrichaceae families. A high percentage of radiation-resistant bacteria from other family were also seen in few zones post radiation. FIG. 2 provides visualization and hierarchal clustering of the samples based upon the top 50 genera. There is clear grouping of the pre-radiation samples (C) and the radiation samples (R). Based upon Unifrac analysis and principal component analysis the pre-radiation (blue) and radiation (red) samples are clearly and significantly (p<0.001) grouped in three dimensional space (FIG. 3). Finally, based upon rarefaction analysis, we see that the predicted diversity of the post radiation samples are of significantly (P=0.002) lower than before radiation.

Data in Table 2 shows that the members of fungal family of Thamnidiaceae are ionizing radiation resistant and present in all the zones, except Zone 1. However, unlike bacteria, different fungi families dominate the soil in different post radiation zones. In Zone 1, the Trichocomaceae family is the most abundant radiation-resistant fungi, whereas in Zone 4, the Hypocreales family is the abundant radiation-resistant fungi. The presence of radiation-resistant fungi in the soil is not surprising considering that previous studies showed the growth of fungi on the nuclear rods and other sources emitting high level of ionizing radiation (14-15).

There are no reports on the microorganisms present in the rhizosphere of trees in the Pine Barrens and their role in promoting plant growth. While this makes a direct conclusion on the sensitivity of the microorganisms to ionizing radiation and their role in vegetation recovery harder, several indirect inferences can be made. The soil of the Pine Barrens is very poor in nitrogen. Nitrogen-fixing microorganisms and the decay of organic material provide the major sources of Nitrogen (16). Combination of the absence of vegetation in the region near the source of radiation and the sensitivity of the microorganisms involved in the nitrogen cycle (Table 1) could make the recovery of the forest harder. The fungi that survived the radiation could also be hindering the colonization of essential microorganisms through competitive exclusion. The presence of fungi in the soil has shown to impact the potential niches for bacteria in several studies (17-18). The soil in the Gamma Forest, similar to that of the Pine Barrens Forests, is rich in iron and aluminum, with an average concentration of 2955 mg/Kg wet soil and 2606 mg/kg wet soil, respectively. The pH of the soil ranges from 4.25 to 5.0. Additional selective pressure includes winter temperatures in which the soil falls below the freezing point. Environmental niches occupied community of resistant microorganisms also could have hindered the re-colonization of the soil by radiation-sensitive microorganisms. These sensitive organisms could have been critical for the plant growth and only upon the arrival of the required microorganisms could plants re-grow. Soil in Zones 1 and 2b still does not have the required microbial community for the growth of any vegetation, including grass. Further studies are being carried out to evaluate these hypotheses.

Without proper microbial remediation an ecosystem that has been exposed to ionizing radiation could take several decades to recover, even after all the ionizing radiation exposure has ceased. Current scientific knowledge does not allow one to elucidate the role of microorganisms and impact of the changes in the microbial community structure over the long term in the recovery of the ecosystem. In the areas where ionizing radiation exposure is likely, such as nuclear reactors or high-level radioactive wastes storage sites, it would be valuable to study and archive the radiation sensitive microbial organisms that are essential for proper ecosystem function. Should the ionizing radiation find its way into such high-risk areas, artificial introduction of the essential microorganisms could allow a faster recovery of the ecosystem.

We determined that the presence of high level of ammonia oxidizing bacteria in soil is critical for the vegetation to grow in Pine Barrens Forest. These bacteria maintain the acidic pH of the soil and keep the nitrogen content low, both of which are essential for vegetation to grow. We found that the members of Nitrosomonadaceae family (primarily Nitrosovibrio genus) are the only bacterial population carrying out ammonia oxidation in Pine Barrens soil. As can be seen in Table 1 and FIG. 1a, there is direct correlation between the presence of higher plants in the Zone and survival of Nitrosomonadaceae to ionizing radiation. In the soil sample obtained from Zones having higher plants (Zones 3, 5 and 6), there was negligible or no decrease in the percentage of these organisms upon exposure to radiation. In contrast, the soil samples from other Zones saw a sharp decline in Nitrosomonadaceae. Upon termination of ionizing radiation, plants including pitch pines and oak were able to grow in the Zones containing higher Nitrosomonadaceae, whereas in other Zones one can expect the plants to grow once the ammonia oxidizers make the soil conducive for growth. Similarly, the absence of other ionizing radiation sensitive organisms whose ecological roles have not yet been recognized could be preventing the growth of any vegetation (including grass) in Zones 1 and 2b. To confirm the sensitivity of Nitrosomonadaceae to ionizing radiation, the soil from Zone 1 was radiated with 4.5 kGy of gamma radiation. The percentage of Nitrosomonadaceae in the soil was reduced to zero (Table 1) along with the appearance for more radiation resistant bacteria and fungi (Table 1 and 2).

While the parameters influencing the reestablishment of Nitrosomonadaceae are not clear, the chemical properties of soil do not seem to be important. The soil in all of the Zones has almost similar in chemistry with pH ranging from 4.25 to 5.0. Average concentrations of iron and aluminum were 2955 and 2606 mg/Kg wet soil, respectively. Several studies have shown that the presence of fungi in the soil could impact the potential niches for bacteria¹⁷⁻¹⁸. The fungi that survived the radiation could competitively exclude organisms required for vegetation growth.

Experimental data points to the importance of microorganisms in ecosystem recovery upon exposure to ionizing radiation. In the areas where ionizing radiation exposure is likely, such as nuclear reactors or high-level radioactive wastes storage sites, it would be valuable to study and archive the radiation sensitive microbial organisms that are essential for proper ecosystem function. Should the ionizing radiation find its way into such high-risk areas, artificial introduction of the essential microorganisms could allow a faster recovery of the ecosystem.

Exemplary Methods

Sample collection: After the removal of surface litter, soil samples were collected from the different Zones of Gamma Forest between the depth of 0 and 20 cm. The surface litter was removed prior to sample collection. While collecting samples, care was taken not to disturb the surrounding areas, and afterward, each sample collection hole was filled back with the soil. All distinguishable debris and pebbles were removed using sterile forceps, and the soil was mixed thoroughly prior to experimentation.

Gamma Radiation exposure: 50 g of soil sample from each zone were collected in a 50 mL polypropylene tube and irradiated with gamma radiation at the Long Island Pine Barrens Forest ionizing irradiation facility within the Department of Biology. A ¹³⁷Cesium isotope with current source activity of 1,576 curies was the source of gamma radiation, and the soil samples were placed 3 inches from the source. The dose rate was 314.36 R/min.

EMA treatment and DNA isolation: The pre-radiated and post-radiated soil samples were treated with DNA cross-linker ethidiummonoazide bromide (EMA) according to Nocker and Camper¹³ within 24 h. EMA treatment allows us to preferentially analyze viable bacteria and reduce the probability of polymerase chain reaction (PCR) amplification of DNA from dead or moribund cells. DNA was isolated using PowerMax™ DNA Isolation Kit (MO BIO Laboratories, Inc., Carlsbad, Calif.).

TEFAP: Data on the microbial communities present in the soil was obtained by carrying out pyrosequencing analysis on the DNA. The microbial tag-encoded FLX amplicon pyrosequencing (TEFAP) was performed using primers Gray28F (GAGTTTGATCNTGGCTCAG) and Gray519r (GTNTTACNGCGGCKGCTG) for bacterial populations and ITS1 (CTTGGTCATTTAGAGGAAGTAA) and ITS4 (TCCTCCGCTTATTGATATGC) were used as primers for fungal populations. Initial generation of the sequencing library utilized a one-step PCR with a total of 30 cycles, a mixture of Hot Start and HotStar high fidelity taq polymerases, and amplicons originating and extending from the forward primers.¹⁹⁻²⁰ Tag-encoded FLX amplicon pyrosequencing analyses utilized Roche 454 FLX instrument with Titanium reagents. Following sequencing, all failed sequence reads, low quality sequence ends and tags and primers were removed along with the sequences collections depleted of any non-bacterial/fungal bacterial/fungal ribosome sequences and chimeras. To determine the identity of microorganisms in the remaining sequences, sequences were denoised, assembled into clusters and queried using a distributed BLAST (www.krakenblast.com) algorithm²³ against a comprehensive database of high quality rDNA sequences derived from NCBI (01-01-11) and evaluated as described previously.^{21-22,24-25} Unifrac analysis²⁶ to generate weighted distance matrices were evaluated using principal component analysis and rarefaction analysis was performed using Mothur²⁷ as described previously.^{21-22,24-25} Two tailed T-test was utilized to evaluate the significance of rarefaction data. Dual hierarchal dendrograms based upon Ward's minimum variance and Manhattan distances were generated using NCSS 2007.

Thus, the present invention is a method for aiding an ecosystem in recovering from the effects of exposure to radiation, comprising introducing microorganisms into the ecosystem. In this method, the microorganisms can replace native microorganisms destroyed, killed, or reduced in number by the exposure to radiation. In one embodiment of the invention, the microorganisms can be introduced into the ecosystem prior to exposure of the ecosystem to radiation to create an overabundance of the microorganisms in anticipation that not all of the microorganisms will be destroyed or killed by the radiation. In another embodiment of the invention, the microorganisms can be reintroduced into the ecosystem after exposure of the ecosystem to radiation to repopulate the ecosystem.

In one example, the native microorganisms in an ecosystem, such as a site surrounding a nuclear reactor facility, can be cataloged. If a nuclear or irradiating accident or event occurs in which the ecosystem is contaminated with radiation, thus killing or destroying the microorganisms, flora, and/or fauna in the ecosystem, native microorganisms can be reintroduced into the ecosystem to aid in the recovery of the ecosystem.

In another example, the native microorganisms in an ecosystem, such as a site surrounding a nuclear reactor facility, can be cataloged. Quantities of the native microorganisms can be introduced into the ecosystem prior to any irradiating accident or event occurring, creating a robust population of the native microorganisms in the ecosystem. If a nuclear or irradiating accident or event then occurs in which the ecosystem is contaminated with radiation, potentially some of the robust population of the native microorganisms may have survived, thus aiding in the recovery of the ecosystem. Additional native microorganisms also can be reintroduced into the ecosystem to aid in the recovery of the ecosystem.

One embodiment of the present invention also is a method for aiding an ecosystem in recovering from the effects of exposure to radiation, comprising the steps of:
a) prior to the ecosystem being exposed to radiation, cataloging and/or preserving the microorganisms in the soil of the ecosystem (referred to as the native microorganisms);
b) after the ecosystem being exposed to radiation, cataloging and/or preserving the native microorganisms in the soil of the ecosystem; and
c) introducing native microorganisms into the ecosystem after the ecosystem has been exposed to radiation.

Another embodiment of the present invention also is a method for aiding an ecosystem in recovering from the effects of exposure to radiation, comprising the steps of:
a) prior to the ecosystem being exposed to radiation, cataloging and preserving the native microorganisms in the soil of the ecosystem; and
b) introducing native microorganisms into the ecosystem prior to the ecosystem being exposed to radiation.

Native microorganisms can include microorganisms that are identical to the native microorganisms, microorganisms that are equivalent to the native microorganisms, microorganisms that behave in a similar manner to the native microorganisms, and microorganisms that can be substituted for the native microorganisms with little or no negative ecological and/or environmental effect.

This method can further comprise cataloging the flora in the ecosystem prior to and after the ecosystem has been exposed to radiation. This method also can further comprising cataloging the fauna in the ecosystem prior to and after the ecosystem has been exposed to radiation.

In this method, the microorganisms introduced into the ecosystem can be microorganisms reduced or eliminated after the ecosystem has been exposed to radiation. Alternatively, in this method, the microorganisms introduced into the ecosystem can be microorganisms equivalent to the microorganisms reduced or eliminated after the ecosystem has been exposed to radiation.

In an embodiment of the invention, the microorganisms are in either vegetative or sporulating form. In another embodiment of the invention, the microorganisms are introduced into the ecosystem to allow recovery of vegetation.

The above detailed description of the preferred embodiments, and the examples, are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention

1. (canceled)

2. (canceled)

3. A method for aiding an ecosystem in recovering from the effects of exposure to radiation, comprising the steps of:
a) prior to the ecosystem being exposed to radiation, collecting samples of soil from the ecosystem and cataloging the microorganisms in either vegetative or sporulating form in the soil of the ecosystem;
b) after the ecosystem being exposed to radiation, cataloging the microorganisms in either vegetative or sporulating form in the soil of the ecosystem; and
c) introducing into the ecosystem microorganisms in either vegetative or sporulating form prior to and/or after the ecosystem has been exposed to radiation.

4. The method as claimed in claim 3, further comprising cataloging the flora in the ecosystem prior to and after the ecosystem has been exposed to radiation.

5. The method as claimed in claim 3, further comprising cataloging the fauna in the ecosystem prior to and after the ecosystem has been exposed to radiation.

6. The method as claimed in claim 3, wherein the microorganisms in either vegetative or sporulating form introduced into the ecosystem are microorganisms destroyed, killed or reduced in number after the ecosystem has been exposed to radiation.

7. The method as claimed in claim 3, wherein the microorganisms introduced into the ecosystem are microorganisms in either vegetative or sporulating form equivalent to the microorganisms destroyed, killed, or reduced in number after the ecosystem has been exposed to radiation.

8. The method as claimed in claim 3, wherein the microorganisms are introduced into the ecosystem to allow recovery of vegetation.

9. The method as claimed in claim 3, further comprising the steps of:
determining the identity of microbial communities present in the soil to determine native microorganisms in the soil; and
introducing the microorganisms into the ecosystem prior to the exposure to radiation to create an overabundance of the microorganisms in anticipation that not all of the native microorganisms will be destroyed, killed, or reduced in number by the radiation.

10. The method as claimed in claim 9, wherein the microorganisms introduced into the ecosystem are selected from the group consisting of microorganisms that are identical to the native microorganisms, microorganisms that are equivalent to the native microorganisms, microorganisms that behave in a similar manner to the native microorganisms, and microorganisms that can be substituted for the native microorganisms with little or no negative ecological and/or environmental effect on the ecosystem.

11. The method as claimed in claim 9, wherein the microorganisms introduced into the ecosystem after the exposure to radiation are to repopulate the ecosystem with the microorganisms.

12. The method as claimed in claim 11, wherein the microorganisms are selected from the group consisting of microorganisms that are identical to the native microorganisms, microorganisms that are equivalent to the native microorganisms, microorganisms that behave in a similar manner to the native microorganisms, and microorganisms that can be substituted for the native microorganisms with little or no negative ecological and/or environmental effect on the ecosystem.
